(19) United States
(12) Patent Application Publication
(10) Pub. No.: US 2007/0218099 A1
(43) Pub. Date: Sep. 20, 2007

(54) PREPARATION AND CHARACTERIZATION OF POLYETHYLENEGLYCOL/POLYESTERS AS BIOCOMPATIBLE THERMO-SENSITIVE MATERIALS

(76) Inventors: Moon Suk Kim, Daejeon (KR); Hai Bang Lee, Daejeon (KR); Hoon Hyun, Jeollabuk-do (KR); Gilson Khang, Daejeon (KR); Jung Won Lee, Jeollabuk-do (KR)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/686,168

(22) Filed: Mar. 14, 2007

(30) Foreign Application Priority Data
Mar. 15, 2006 (KR) 10-2006-0023991

(51) Int. Cl. A61F 2/02; C08F 20/00

(52) U.S. Cl. 424/423; 525/438

(57) ABSTRACT

The present invention relates to a biocompatible and thermosensitive poly(ethylene glycol)/polyester block copolymer and a method of its preparation thereof, and particularly to a multi-functional intelligent hydrogel polymer comprising a hydrophilic part of a poly(ethylene glycol) (PEG) having a low molecular weight and a hydrophobic part comprising an ester-based caprolactone (CL) segment as an essential ingredient and further comprising a para-dioxanone (PDO) segment, a trimethylene carbonate (TMC) segment or a PDO/TMC copolymer containing the PDO and the TMC segments in a predetermined ratio, which easily forms a desired-shaped gel and decomposes or disperses without necessitating the operation process for removing the gel due to the temperature-dependent phase transition caused by the coagulation and the expansion of polymer micelles comprising a hydrophilic part and a hydrophobic part, thus being applicable to a drug delivery system or a porous support for tissue engineering purpose.

### TECHNICAL FIELD

The present invention relates to a biocompatible and thermosensitive poly(ethylene glycol)/polyester block copolymer and the preparation method thereof, and particularly to a multi-functional intelligent hydrogel polymer comprising a hydrophilic part of a poly(ethylene glycol) (PEG) having a low molecular weight and a hydrophobic part comprising an ester-based caprolactone (CL) segment as an essential ingredient and further comprising a para-dioxanone (PDO) segment, a trimethylene carbonate (TMC) segment or a PDO/TMC copolymer containing the PDO and the TMC segments in a predetermined ratio, which easily forms a desired-shaped gel and decomposes or disperses without necessitating the operation process for removing the gel due to the temperature-dependent phase transition caused by the coagulation and the expansion of polymer micelles comprising a hydrophilic part and a hydrophobic part, thus being applicable to a drug delivery system or a porous support for tissue engineering purpose.

### RELATED PRIOR ART

Numerous researches have been made in the field of hydrogel since the advent of hydrogel was first prepared using PHEMA (polyhydroxyethyl methacrylate) in 1960. The introduction of hydrogel prepared using calcium alginate toward 1980 was a remarkable turning point in the biological material field. Since then, the synthesis of hydrogel for biological material using natural or synthetic polymer has made rapid progress. One of the most representative features of the hydrogel is a swelling property conferred by the network structure of hydrophilic polymer that may absorb a large amount of water. The three dimensional network structure of hydrogel may be formed by various factors such as a covalent bond, a hydrogen bond, van der Waals bond or physical cohesion. Hydrogel may inhibit the denaturalization due to enzyme or pH in the intestines, and may also be endowed with a property of releasing drugs at a stimulus in human body. Due to the stimulus-response sensor-like behavior, the hydrogel may cause reversible volumetric or sol-gel transition within a few minutes triggered by any stimulus in human body.

Sources of the stimulus may be divided into a physical stimulus such as temperature, electricity, solvent change, light, pressure, sound and magneticity and a chemical stimulus such as ion and recognition of a certain molecule. These stimulus-response hydrogels are expected to be applied to an efficient and controlled-release drug delivery system that may minimize the side effect of drugs. Further, stem cells drawing attention nowadays in the field of regenerative medicine are reported to differentiate into various tissues by the activity of cytokines. Considering these researches, stimulus-response hydrogel in combination of cells, genes and stem cells is expected to induce the generation of various artificial organs such as cartilage, bones and bloods.

A temperature-stimulated hydrogel, which pertains to the present invention, is most widely used in drug delivery system or cell delivery system for tissue regenesis. It is because various polymers show temperature transition property. By the introduction of hydrophilic groups that may enable a polymer to be dissolved or to swell, the solubility of general polymer in water increases. However, a polymer that comprises both hydrophilic and hydrophobic groups such as methyl, ethyl and propyl groups show a decrease in water solubility with the increase of temperature, thus having a low critical solution temperature (LCST). A hydrogel, which is prepared using a polymer with LCST like a poly(ethylene glycol)/biodegradable polyester copolymer according to the present invention, is a [symbol] (?) hydrogel that contracts when a temperature is elevated higher than LCST. A polymer comprising hydrophilic and hydrophobic parts is of a sol phase because the polymer may be dissolved in water due to dominant hydrogen bonding between the hydrophilic group and water molecule. However, the hydrophobic bonding becomes more dominant than the hydrogen bonding with the increase of temperature, thus causing aggregation of the hydrophobic part and resulting in phase transition into gel state. Therefore, the increase of hydrophobic portion in a polymer lowers LCST, which means that LCST may be adjusted by controlling hydrophilic and hydrophobic chains. This polymer is a sol sate at normal temperature and flows like fluid, and the encapsulation of drugs may attained by a simple mixing, whereas the polymer hydrogel become in a gel state and shows a controlled release behavior when heat is applied higher than body temperature. This shows swelling-shrinking behavior when crosslinked, while it shows sol-gel phase transition when non-crosslinked. Poly(N-isopropylacrylamide) is most widely used because it has a LCST around body temperature, and its copolymers in combination with butylmethacrylate, poly(ethylene glycol), poly(propyl glycol) is being used in human body through sol-gel transition in various temperature ranges. Poly(ethylene oxide)-poly(propylene oxide) copolymer (PEO-PPO) also shows a sol-gel transition behavior and is widely used under the various trademarks such as Pluronic, Poloxamers, Tetronic [U.S. Pat. No. 4,188,373].

Meanwhile, the sol-gel polymer should be released from human body by metabolism after being used in human body. In this respect, many patents disclose sol-gel polymer that contains polylactide-polyglycolide copolymer (PLGA) in hydrophobic part as a biodegradable polymer [U.S. Pat. Nos. 4,882,168, 4,716,203, 4,942,035, 5,476,909 and 5,548,035].

There have been attempts made to develop an intelligent hydrogel that may be applied to drug delivery system and tissue engineering using a physiochemical property of stimulus-response polymer. To be applied to drug delivery system and tissue regenesis for injection formulation, this intelligent hydrogel should have low viscosity, fast gelation property, biodegradability and low molecular weight to be easily released from human body. Further, to serve as a biological material, this should also be biocompatible and should not damage cells and other organs during decomposition or release from human body.

In order to apply a thermosensitive hydrogel to a drug delivery system for injection formulation or a porous support for tissue engineering, the present inventors have finally completed the present invention (i) by using poly(ethylene glycol) as hydrophilic polymer, which is highly soluble in water and organic solvent, non-cytotoxic, shows no immunorejection response and enables to increase the amount of water that a copolymer with hydrophobic biodegradable ester-based polymer to absorb when applied to human body, thus being capable of controlling the decomposition period, (ii) by using a hydrophobic polymer, which is obtained by polymerizing a biocompatible and biodegradable ester-based caprolactone (CL) with a para-dioxanone (PDO), a trimethylene carbonate (TMC) or both the PDO and the TMC in a certain ratio, which may release from human body after being biologically metabolized through dissolution, chemical hydrolysis and enzyme-related decomposition and may also control the decomposition period by means of the control of molecular weight and chemical composition, (iii) by investigating a temperature-dependent or a concentration-dependent sol-gel phase transition behavior in aqueous solution through the change of the kind of hydrophobic group and chemical structure, and (iv) by ascertaining that a sol-phase copolymer injected in a mouse may induce the gel formation at a temperature near the body temperature.

Therefore, the present invention aims to provide a biocompatible and thermosensitive poly(ethylene glycol)/biodegradable polyester block copolymer with a molecular weight of 2,000-7,000 g/mole, which comprises a hydrophilic part and a hydrophobic part, where the hydrophilic part comprises a poly(ethylene glycol) and the hydrophobic part comprises a caprolactone (CL) segment as an essential ingredient and further comprises a para-dioxanone (PDO) segment, a trimethylene carbonate (TMC) segment or both the PDO and the TMC segments.

### DETAILED DESCRIPTION OF INVENTION

The present invention relates to a biocompatible and thermosensitive poly(ethylene glycol)/biodegradable polyester block copolymer, which comprises a hydrophilic part and a hydrophobic part, where the hydrophilic part comprises a poly(ethylene glycol) and the hydrophobic part comprises a caprolactone (CL) segment as an essential ingredient and further comprises a para-dioxanone (PDO) segment, a trimethylene carbonate (TMC) segment or both the PDO and the TMC segments. The molar ratio of the caprolactone segment is 50-95, and the molar ratio of the PDO or the TMC is 50-5 relative to the hydrophobic part.

The present invention relates to a biocompatible and thermosensitive [symbol] poly(ethylene glycol)/biodegradable polyester block copolymer having a molecular weight of 2,000-7,000 g/mole and the preparation method thereof.

Further, the present invention relates to a drug delivery system for an injection formulation or a porous support for tissue engineering, which comprises the poly(ethylene glycol)/biodegradable polyester block copolymer herein.

Hereunder is provided a detailed description of the present invention.

The present invention relates to a block copolymer of the present invention comprises a hydrophilic part of a poly(ethylene glycol) (PEG) having a low molecular weight and a hydrophobic part comprising an ester-based caprolactone (CL) segment as an essential ingredient and further comprising a para-dioxanone (PDO) segment, trimethylene carbonate (TMC) segment or a copolymer comprising the PDO and TMC in a predetermined ratio. The block copolymer serves as a multi-functional intelligent hydrogel polymer, which easily forms a desired-shaped gel and decomposes or disperses without necessitating the operation process for removing the gel due to the temperature-dependent phase transition caused by the coagulation and the expansion of polymer micelles comprising a hydrophilic part and a hydrophobic part, thus being applicable to a drug delivery system or a porous support for tissue engineering purpose.

The poly(ethylene glycol) (PEG) used as an initiator in the present invention has various advantages in a drug delivery and a tissue engineering field because it easily captures and releases drugs and shows high solubility in water and organic solvent and superior biocompatibility without exhibiting toxicity and immunorejection response. Approved by FDA, PEG has been widely used in the manufacture of medicine. Further, among hydrophilic polymers, PEG is highest in inhibiting protein adsorption and improves the biocompatibility of blood-contacting material, thus widely being applied as biological material. However, the difficult biodecomposition of PEG-containing biological material has been raised as a problem. PEG is accumulated in human body instead of being decomposed, and is known to increase plasma cholesterol and cytotoxcity of neutral fat after peritoneal injection. Therefore, to overcome these problems, the present inventors have finally prepared a poly(ethylene glycol)/biodegradable polyester block copolymer herein by copolymerization of PEG having a molecular weight of lower than 5,000 g/mole, which is easily removed from human body through the filtration of the kidney, and a biodegradable ester-based monomer, which may be metabolized into biocompatible metabolites.

The ester-based biodegradable polymer is advantageous in that the time required for being decomposed may be controlled by modifying the molecular weight and chemical content. The block copolymer of poly(ethylene glycol) (PEG) and polycaprolactone (PCL), which is used as a basic model in the present invention, is already applied as a biological thermosensitive copolymer showing a sol-gel phase transition property. However, although caprolactone is biodegradable and compatible with various polymers and easily crystallizes, the high crystallinity reduces the biocompatibility with tissues and shows long-term decomposing behavior.

Thus, the present invention reduces the crystallinity and controls the biodecomposition period by mixing caprolactone with biodegradable ester-based para-dioxanone (PDO), trimethylene carbonate (TMC) or both the PDO and the TMC in a predetermined mixing ratio. That is, the hydrophobic part represented by the following Formula 1, and each segment is randomly copolymerized.

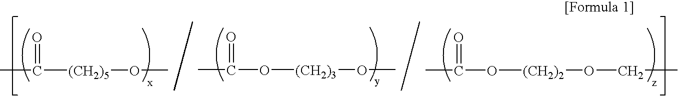

wherein each of x, y and z is a segment that constitutes the hydrophobic polyester part; x is 50-95 mol %; and (y+z) is 5-50 mol % (including the case that y or z is zero).

A block copolymer of the present invention by performing a ring-opening copolymerizing of an ester-based caprolactone (CL) with a para-dioxanone (PDO), trimethylene carbonate (TMC) or both the PDO and the TMC using poly(ethylene glycol) as a hydrophilic part having a low molecular weight (Mn=350-2,000 g/mole). After poly(ethylene glycol) was subject to azeotropic distillation and dried, an ester-based monomer was added, and methylene chloride (MC) or toluene was also added as a solvent. Polymerization is performed at a temperature of from −40 to 130° C. by the addition of an acid catalyst as a monomer activator. As the acid catalyst, at least one selected among HCl, HBr, CF₃COOH, CCl₃COOH, BrCH₂COOH, CH₃COOH, BCl₃, BBr₃ and camphorsulfonic acid (camphorsulfonic acid) is preferred.

The aqueous phase of the synthesized poly(ethylene glycol)/biodegradable polyester block copolymer prepared in the present invention shows a novel thermosensitive sol-gel phase transition in that it maintains sol state with flowing property at room temperature, forms gel at a certain temperature range (30-46° C.), and restores the flowing property at a higher temperature than critical temperature (44-47° C.). Thermal property and crystallinity are observed by using a differential scanning calorimetry (DSC) and X-ray diffraction metry. The formation and maintenance of gel is ascertained by injecting a block copolymer of the present invention in a mouse.

Further, for serving as a drug delivery system for an injection formulation or as a functional support for regenerating tissue, this thermosensitive block copolymer should have a low viscosity, a fast gel formation and a low molecular weight to be easily released from human body. In the present invention, viscosity may be lowered by lowering crystallinity by the introduction of ester-based para-dioxanone (PDO), trimethylene carbonate (TMC) or both the PDO and the TMC in a predetermined amount to caprolactone. Further, a molecular weight similar to an expected value may be obtained in the present invention, thus satisfying another requirement for biocompatible and thermosensitive hydrogel, i.e. low molecular weight.

It is preferred to synthesize poly(ethylene glycol)/biodegradable polyester block copolymer with a total molecular weight of 2,000-7,000 g/mole. If the total molecular weight is less than 2,000 g/mole, a sol-gel phase transition at a temperature near body temperature, which is a purpose of the present invention, may not be induced and a sol phase is maintained instead. If a total molecular weight is higher than 7,000 g/mole, the biodecomposition may take a long period of time due to the large molecular weight.

Representative example of a poly(ethylene glycol)/biodegradable polyester block copolymer according to the present invention may be represented by the following Scheme 1.

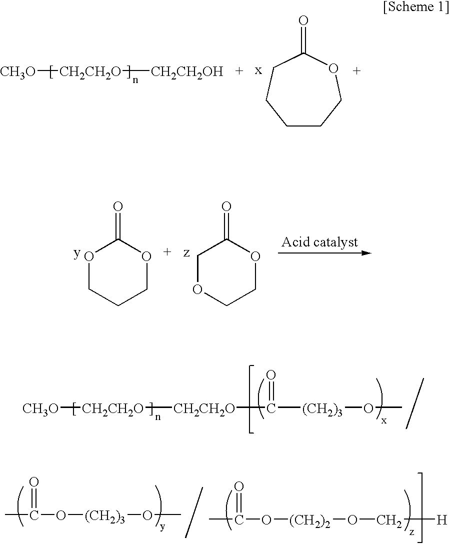

In the Scheme 1, n is a repeating unit of poly(ethylene glycol) that constitutes a hydrophilic part; each of x, y and z is a segment that constitutes a hydrophobic polyester, respectively; and x is 50-95 mol % and (y+z) is 5-50 mol % (including the case where y or z is zero).

As compared to a reference model, i.e. poly(ethylene glycol)-polycaprolactone block copolymer, thus prepared poly(ethylene glycol)/biodegradable polyester block copolymer has the following properties: a lower crystallinity and such a low viscosity as to be easily handled; a fast gel formation; and such a lower molecular weight to be easily released from human body, thus being capable of serving as a drug delivery system for an injection formulation or as a functional support for regenerating tissue. Further, the biodegradation period may be controlled by adding a biodegradable ester-based para-dioxanone (PDO), trimethylene carbonate (TMC) or both the PDO and the TMC to a caprolactone showing a long-term biodegradability. Moreover, a poly(ethylene glycol)/biodegradable polyester block copolymer aqueous solution according to the present invention shows may widely variable temperature-dependent sol-gel phase transition behavior by appropriately using a biodegradable polymer such as para-dioxanone (PDO) and/or trimethylene carbonate (TMC). Thus, a block polymer of the present invention may also satisfy the gelation at a temperature higher or lower than body temperature, let alone at a temperature near body temperature when applied to human body as a biological material, which is the object of the present invention.

### BRIEF DESCRIPTION OF DRAWINGS

FIG. 1 is a ¹H-NMR spectrum of methoxypoly(ethylene glycol)-(polycaprolactone-co-polytrimethylene carbonate) block copolymer prepared in Example 1 according to the present invention.

FIG. 2 is a ¹H-NMR spectrum of methoxypoly(ethylene glycol)-(polycaprolactone-co-polypara-dioxanone) block copolymer prepared in Example 2 according to the present invention.

FIG. 3 shows crystalline peaks of block copolymers prepared in Examples 1-2 according to the present invention analyzed with an X-ray diffraction meter.

FIG. 4 shows a sol-gel phase transition behavior in an aqueous solution of block copolymers prepared in Examples 1-2 according to the present invention. (A) is a sol-state photograph taken at 25° C.; (B) shows a phase transition to a gel state at 37° C.; and (C) shows a sol-state copolymer aqueous solution spouted through a syringe.

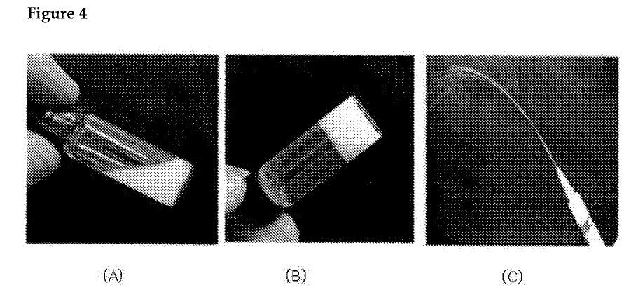

FIG. 5 shows a sol-gel phase transition behavior obtained by measuring the temperature dependent viscosity of block copolymers prepared in Examples 1-2 according to the present invention. (A) shows a dependency of viscosity at a gelation temperature on the concentration of MPEG-PCL block copolymer. (B) shows a sol-gel temperature and a viscosity of a block copolymer prepared according to the present invention.

FIG. 6 is a result of an animal experiment according to the present invention Example 4. The photographs show in-vivo gelation after subcutaneously injecting a mixture of sol-phase block copolymer aqueous solution prepared in Examples 1-2 with bovine serum albumin in a mouse. (A) shows the subcutaneous injection of the sol-phase polymer solution in a mouse. (B) shows the gel formation in the subcutaneously injected region. (C) shows the gel separated from the mouse.

FIG. 7 is a result of an animal experiment according to the present invention Example 5. A sol-state aqueous solution of block copolymer prepared in Examples 1-2 was mixed with different amounts of dexamethasone and bone marrow stem cells (BMSCs) and subcutaneously injected in a mouse. The formed gel was stained by von Kossa, and the bone formation for tissue engineering was ascertained through photographs 4 weeks after the injection. (A) shows a result of transplantation of hydrogel only. (B) shows a result of transplantation of a mixture of hydrogel and bone marrow stem cells. (C) shows a result of transplantation of a mixture of hydrogel, bone marrow stem cells and 1 mg of dexamethasone. (D) shows a result of transplantation of a mixture of hydrogel, bone marrow stem cells and 5 mg of dexamethasone. (E) shows a result of transplantation of a mixture of hydrogel, bone marrow stem cells and 10 mg of dexamethasone

### EXAMPLES

The present invention is described more specifically by the following Examples. Examples herein are meant only to illustrate the present invention, and they should not be construed as limiting the scope of the claimed invention.

### Example 1

### Preparation of methoxypoly(ethylene glycol)-(polycaprolactone-co-polytrimethylene carbonate) Block Copolymer [MPEG-PCL/PTMC]

For preparing MPEG-PCL/PTMC block copolymer having a molecular weight of 3,150 g/mole, 1.7 g (2.26 mmol) of methoxypoly(ethylene glycol) (MPEG) as an initiator and 80 mL of toluene were placed in a well-dried round flask (100 mL), and an azeotropic distillation was performed for 3 hours at 130° C. using a Dean-Stark trap. After the distillation, toluene was completely removed, and methoxypoly(ethylene glycol) (MPEG) was cooled down to room temperature. 5.15 Gram (2.26 mmol) of pre-distilled caprolactone (CL) and 0.27 g (2.25 mmol) of trimethylene carbonate (TMC) were added, and 25 mL of pre-distilled methylene chloride (MC) was also added as a reaction solvent. After 4 mL of HCl was added as a polymerization catalyst, the solution was stirred for 24 hours at room temperature. All the steps were performed under high purity nitrogen. After the reaction was completed, to remove unreacted monomer or initiator, the reaction solution(?) was slowly dropped in 600 mL of hexane and 150 mL of methanol, thus causing precipitation. The precipitates were dissolved in methylene chloride (MC) and filtered through a filter paper. Solvent was removed by using a rotary evaporator, and the filtered precipitates were dried under reduced pressure.

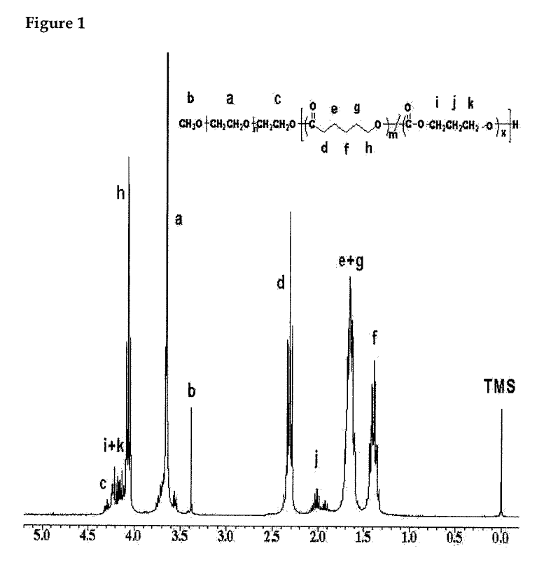

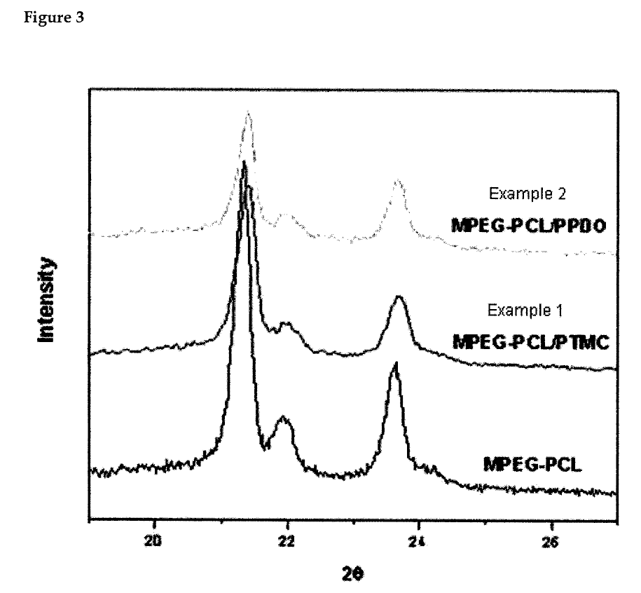

Molar molecular weight of thus prepared copolymer was measured by using ¹H-NMR, and the result was 3,170 g/mole (FIG. 1), which is similar to a theoretically anticipated value. Polydispersity was measured by using a gel permeation chromatography (GPC), and the resultant polydispersity was very sharp (1.19). Crystallinity measured with an X-ray diffraction (XRD) (FIG. 3, 26%) was lower than that of a standard model MPEG-PCL block copolymer (37%), which shows the crystallinity was reduced by the introduction of trimethylene carbonate (TMC).

### Example 2

### Preparation of methoxypoly(ethylene glycol)-(polycaprolactone-co-polypara-dioxanone) block copolymer [MPEG-PCL/PPDO]

For preparing MPEG-PCL/PPDO block copolymer having a molecular weight of 3,150 g/mole, 1.67 g (2.24 mmol) of methoxypoly(ethylene glycol) (MPEG) as an initiator and 80 mL of toluene were placed in a well-dried round flask (100 mL), and an azeotropic distillation was performed for 3 hours at 130° C. using a Dean-Stark trap. After the distillation, toluene was completely removed, and methoxypoly(ethylene glycol) (MPEG) was cooled down to room temperature. 5.11 Gram (2.24 mmol) of pre-distilled caprolactone (CL) and 0.38 mL (2.24 mmol) of para-dioxanone (PDO) were added, and 25 mL of pre-distilled methylene chloride (MC) was also added as a reaction solvent. After 4 mL of HCl was added as a polymerization catalyst, the solution was stirred for 24 hours at room temperature. All the steps were performed under high purity nitrogen. After the reaction was completed, to remove unreacted monomer or initiator, the reaction solution(?) was slowly dropped in 600 mL of hexane and 150 mL of heptane, thus causing precipitation. The precipitates were dissolved in methylene chloride (MC) and filtered through a filter paper. Solvent was removed by using a rotary evaporator, and the filtered precipitates were dried under reduced pressure.

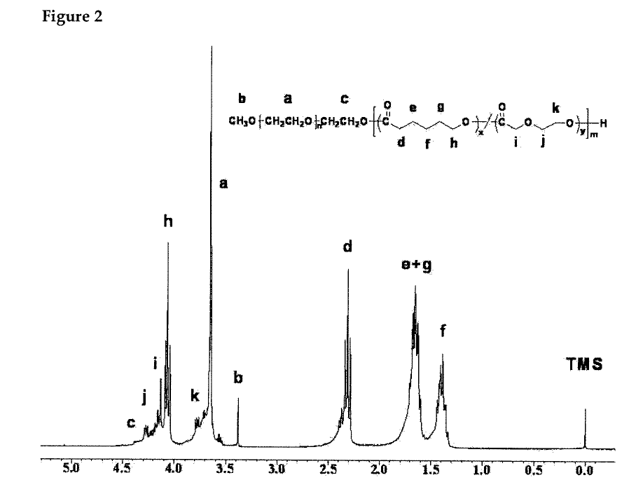

Molar molecular weight of thus prepared copolymer was measured by using ¹H-NMR, and the result was 3,200 g/mole (FIG. 2), which is similar to a theoretically anticipated value. Polydispersity was measured by using a gel permeation chromatography (GPC), and the resultant polydispersity was very sharp (1.17). Crystallinity measured with an X-ray diffraction (XRD) meter (FIG. 3, 27%) was lower than that of a standard model MPEG-PCL block copolymer (37%), which shows the crystallinity was reduced by the introduction of para-dioxanone (PDO).

### Example 3

### Measurement of Sol-Gel Phase Transition Behavior of poly(ethylene glycol)/biodegradable Polyester Block Copolymer as a Function of Time in an Aqueous Solution

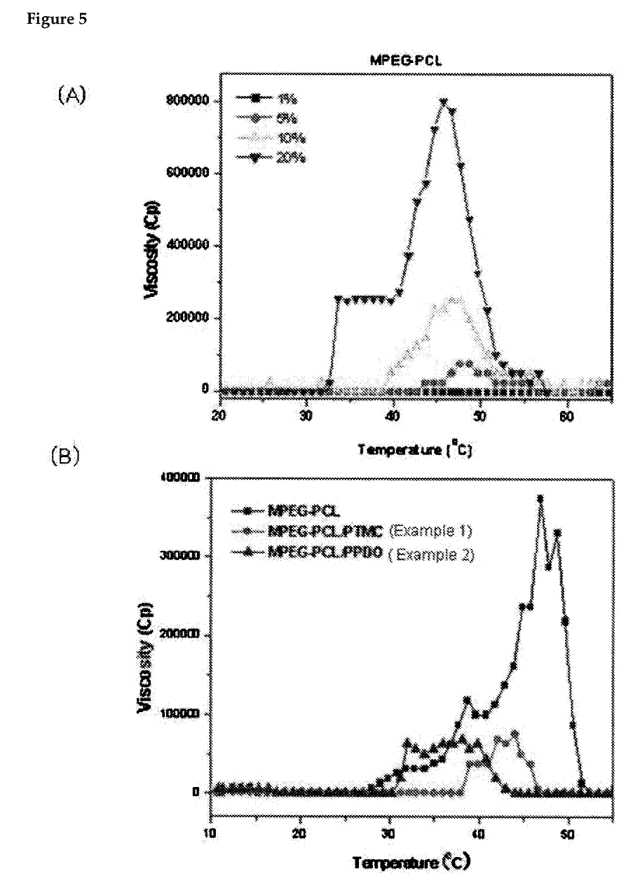

To observe the phase transition behavior of poly(ethylene glycol)/biodegradable polyester copolymer as a function of temperature, each of the synthesized copolymer was dissolved in distilled water into the concentration of 20 wt %, and cold-stored at 4° C. for a day to maintain the equilibrium of uniformly dispersed polymer. The sol-gel phase transition behavior of thus prepared polymer solution was measured with a viscometer by elevating the temperature (1° C. per 3 minutes) from 10° C. to 55° C. at a fixed spin rate of 0.2 rpm (FIG. 5).

### Example 4

### Formation of in vivo Gel of poly(ethylene glycol)/biodegradable Polyester Block Copolymer

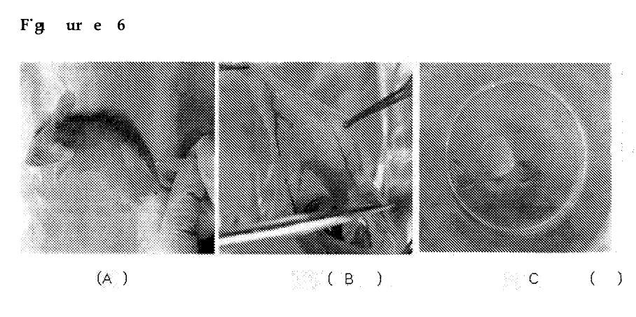

To observe the sol-gel transition behavior around body temperature, the poly(ethylene glycol)/biodegradable polyester block copolymer solution was maintained in a sol phase at room temperature, and 1 mL of the solution was injected under the skin of a mouse with a disposable syringe. After 24 hours, the injected region was cut and the gel formation was ascertained. This shows that the poly(ethylene glycol)/biodegradable polyester block copolymer solution forms a gel fast in human body and that the gel phase is maintained for a long period of time (FIG. 6).

### Example 5

### Formation of Bone for Tissue Engineering Purpose in Gel

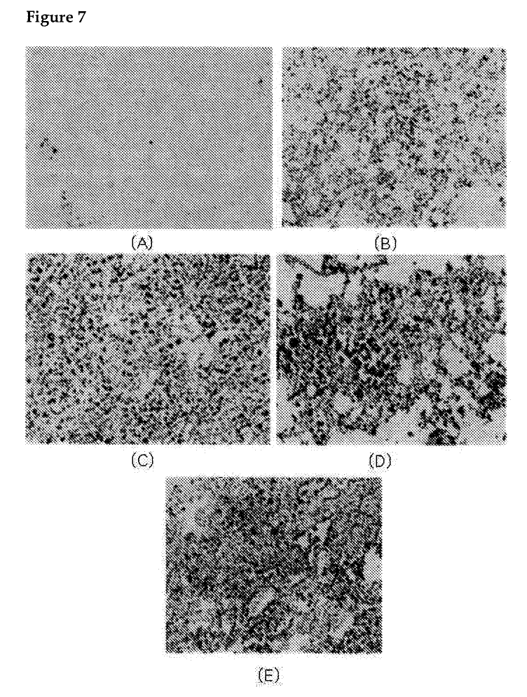

To ascertain that the hydrogel may serve as a tissue engineering support, the formation of tissue engineering bone was observed under an in vivo condition. Different amounts of dexamethasone and bone marrow stem cells (BMSCs) were mixed in hydrogel, and subcutaneously transplanted in a mouse in an injection formulation. The transplanted hydrogel formed gel simultaneously with the injection. The injected region was cut after 4 weeks, and the gel was separated and fixed to 10% formalin. The formalin-fixed specimen was embedded in a paraffin block, and cut into the thickness of 3 μm. The cut specimen was attached to a slide and subject to H&E, von Kossa and osteocalcin staining. The staining results ascertain that the bone formation for tissue engineering was caused by a mixture of hydrogel, dexamethasone (an active ingredient for osteoformation) and bone marrow stem cells. Therefore, the present experiment ascertains that hydrogel may serve as an in vivo support for tissue engineering [FIG. 7].

As described above, an aqueous solution of poly(ethylene glycol)/biodegradable polyester block copolymer according to the present invention may easily contain drugs or biologically active ingredient depending on the temperature change, thus being applicable to a drug delivery system for injection formulation. A block copolymer according to the present invention may act as matrix that controls the drug diffusion due to the biodegradable and biocompatible property, and may also be dissolved in human body by hydrolysis, thus being capable of controlling the drug release behavior or rate. Further, a biodegradable polymer is widely used as various supports for growing cells or tissues in or outside the body. The function of the biodegradable polymer is mainly to provide a place where cells may adhere, move and grow. Therefore, a poly(ethylene glycol)/biodegradable polyester block copolymer according to the present invention may be applied to as a porous support for tissue engineering containing both cells and drugs due to the biodegradable and temperature-sensoring property.

What is claimed is:

1. A biocompatible and thermosensitive poly(ethylene glycol)/biodegradable polyester block copolymer, which comprises a hydrophilic part and a hydrophobic part and has a molecular weight of 2,000-7,000 g/mole, wherein the hydrophilic part comprises a poly(ethylene glycol) and the hydrophobic part comprises a caprolactone (CL) segment as an essential ingredient and further comprises a para-dioxanone (PDO) segment, a trimethylene carbonate (TMC) segment or both the PDO and the TMC segments.

2. The block copolymer of claim 1, which is in a sol phase at room temperature.

3. The block copolymer of claim 1, wherein the poly(ethylene glycol) has a molecular weight of 350-2,000 g/mole.

4. The block copolymer of claim 1, wherein the hydrophobic part is represented by the following Formula 1, and each segment is randomly copolymerized; 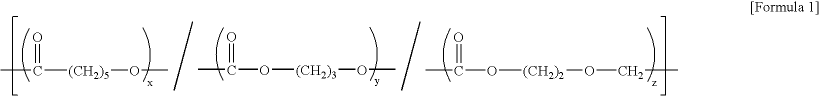
wherein each of x, y and z is a segment that constitutes the hydrophobic polyester part; x is 50-95 mol %; and (y+z) is 5-50 mol % (including the case that y or z is zero).

5. A method for preparing a biocompatible and thermosensitive poly(ethylene glycol)/biodegradable polyester block copolymer, which comprises the step of polymerizing (i) a poly(ethylene glycol) having a molecular weight of 350-2,000 g/mole and (ii) an ester-based monomer comprising a para-dioxanone (PDO) monomer, a trimethylene carbonate (TMC) monomer or both the PDO and the TMC monomers and further comprising a caprolactone (CL) monomer as an essential ingredient within such a range that total molecular weight may be 2,000-7,000 g/mole in the presence of an acid catalyst at a temperature of from −40 to 130° C., whereby preparing a poly(ethylene glycol)/biodegradable polyester block copolymer having a molecular weight of 2,000-7,000 g/mole.

6. The method of claim 5, wherein the acid catalyst is at least one selected from the group consisting of HCl, HBr, CF₃COOH, CCl₃COOH, BrCH₂COOH, CH₃COOH, BCl₃, BBr₃ and camphorsulfonic acid.

7. A drug delivery system for an injection formulation, which comprises the copolymer according to claim 1 that maintain a sol phase at room temperature.

8. A porous support for tissue engineering, which comprises the copolymer according to claim 1.
